(19) United States
(12) Patent Application Publication
(10) Pub. No.: US 2017/0051250 A1
(43) Pub. Date: Feb. 23, 2017

(54) Carbonized Material And Method Of Inducing Cell Differentiation With The Same

(71) Applicant: Linkwin Technology Co., Ltd., Taichung (TW)

(72) Inventor: Yi-Ching Cheng, Taichung (TW)

(21) Appl. No.: 14/830,101

(22) Filed: Aug. 19, 2015

(51) Int. Cl. C12N 5/0793

(52) U.S. Cl. CPC C12N 5/0619; C12N 2533/30; C12N 2537/00; C12N 2535/00

(57) ABSTRACT

A method of inducing cell differentiation with a carbonized material, including the steps of: providing a fiber woven fabric, carbonizing the fiber woven fabric with high temperature to produce a carbonized material, and seeding a cell on the carbonized material where the fiber woven fabric includes a polyacrylonitrile fiber, the carbonized material includes a plurality of carbon fibers, wherein each of the plurality of carbon fibers includes a main body and a plurality of pillars located on a surface of the main body, and are piled up with randomly arranged axes oriented in various directions, and through attachment to the plurality of pillars during the carbonization process, the cell is induced to differentiate into a specialized cell.

### BACKGROUND OF THE INVENTION

1. Technical Field

The present invention relates generally to cell differentiation, and more particularly to a carbonized material and a method of inducing cell differentiation with the carbonized material.

2. Description of Related Art

Due to remarkable advances in neuroscience research, various causes of cerebral neuropathy are discovered. For example, in Parkinson's disease (PD) patients, dopamine, a neurotransmitter in the central nervous system (CNS), is insufficient because dopamine releasing (dopaminergic) neurons in the CNS are dysfunctional or dying. Dopamine is essential for basal ganglia to modulate human motor functions. Therefore, decreasing in the amount of dopamine causes motor coordination impairment.

In the early stages of PD, patients may take dopamine agonists to make up the lack of dopamine, and then retard the damage on motor functions. However, for patients in severe stages of PD, dopamine agonists' concomitant side effects overshadow any cure of the disease. As a result, surgery may be the only cure available in such cases. One kind operation called cell replacement therapy transplants cells that acquire dopaminergic phenotype in the brain of PD patients to substitute dysfunctional neurons, or provides bridges for cell regeneration to restore the pathway in a nervous system.

The cells acquiring dopaminergic phenotype are clinically differentiated from human neural stem cells (hNSCs). Typically, hNSCs are cultured on commercial two-dimensional (2D) tissue culture dishes coated with polystyrene in the process of differentiation. Unfortunately, however, only about 2% of the cells cultured by the latter method have dopaminergic properties. Even after adding in differentiation factor (DF), only at most about 18% of hNSCs may be differentiated into dopaminergic neurons in entire populations of hNSCs. From the above, though cell replacement therapy does avoid side effects caused by dopamine agonists, the cell differentiation rate still has room for improvement.

### BRIEF SUMMARY OF THE INVENTION

In view of the above, the primary objective of the present invention is to provide a carbonized material and a method of inducing cell differentiation with the carbonized material. The carbonized material mimics in vivo environment with the three-dimensional (3D) stacking structure thereof as a 3D scaffold to improve cell differentiation.

The present invention provides a method of inducing cell differentiation with a carbonized material. The method of inducing cell differentiation includes the following steps: provide a fiber woven fabric, carbonize the fiber woven fabric with high temperature to produce a carbonized material, and seed a cell on the carbonized material. The fiber woven fabric includes a polyacrylonitrile fiber, and is carbonized in an environment, which has a pressure between 100 torr and 1 atmospheric pressure and a temperature between 500 and 1200° C., for 1 to 25 minutes. An inert gas is supplied to the environment during the carbonization process. The carbonized material includes a plurality of carbon fibers, wherein each of the plurality of carbon fibers includes a main body and a plurality of pillars; the plurality of pillars are located on a surface of the main body, and are piled up with axes thereof arranged in various directions. Conditions provided by the three dimensional configuration of the carbonized materials imparts to the cell seeded thereon an ability to differentiate into a specialized cell which has a particular function. Thus, through the attachment to the plurality of pillars during the carbonization process, the cell is induced to differentiate into the specialized cell.

The present invention further provides a carbonized material as a substrate of cell differentiation, which is made by a method including the following steps: provide a fiber woven fabric, and carbonize the fiber woven fabric with high temperature to produce a carbonized material. The fiber woven fabric includes a polyacrylonitrile fiber, and is carbonized in an environment, which has a pressure between 100 torr and 1 atmospheric pressure and a temperature between 500 and 1200° C., for 1 to 25 minutes. An inert gas is supplied to the environment during the carbonization process. The carbonized material includes a plurality of carbon fibers, wherein each of the plurality of carbon fibers includes a main body and a plurality of pillars. The plurality of pillars are located on a surface of the main body, and are piled up with axes thereof arranged in various directions.

The carbonized material of the present invention thus provides a substrate for effectively improving cell differentiation rate in an application for providing a heretofore unknown medical treatment application thereof.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a flow chart of a preferred embodiment of the present invention, showing the method of inducing cell differentiation with the carbonized material;

FIG. 2 is a perspective view of the preferred embodiment of the present invention, showing the scanning electron microscopic (SEM) image of the carbonized material;

FIG. 3 is a perspective view of the preferred embodiment of the present invention, showing the SEM image of the pillars;

FIG. 4 is a schematic diagram of the preferred embodiment of the present invention;

FIG. 5 is a perspective view of conventional attachment of hNSCs on a 2D tissue culture substrate; and

FIG. 6 is a schematic diagram of the preferred embodiment of the present invention, showing attachment of hNSCs on a 3D carbonized material which induces cell differentiation.

### DETAILED DESCRIPTION OF THE INVENTION

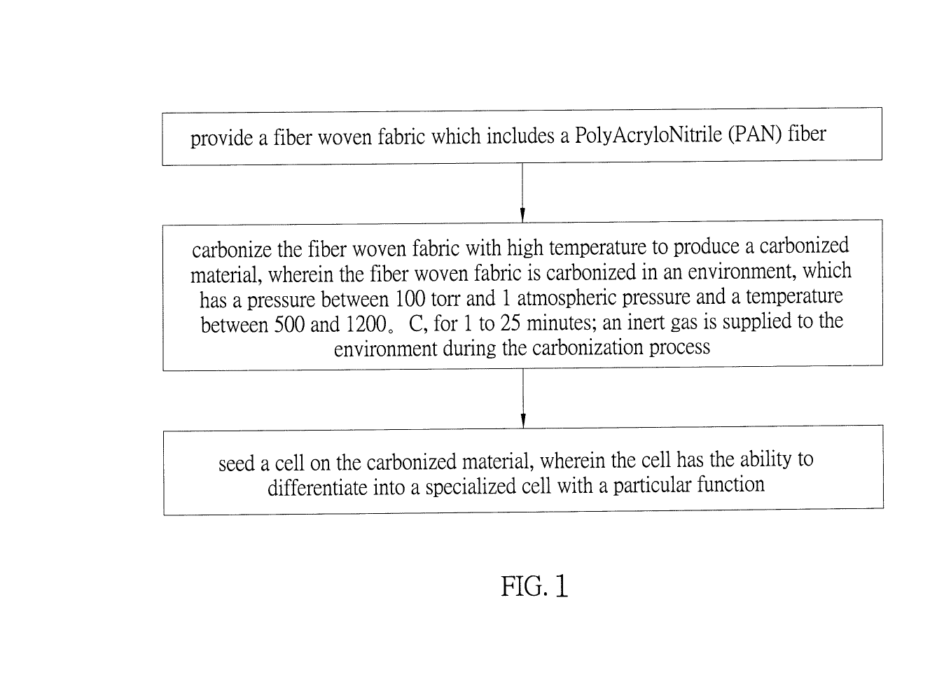

As shown in FIG. 1, a flow chart of a method of inducing cell differentiation with a carbonized material 100 is represented.

The carbonized material 100 includes a carbon fiber fabric, of which the carbonization apparatus and the manufacturing method are disclosed in Taiwan patent I445661, wherein the method of manufacturing the carbonized material 100 will be briefly explained herein, including the steps below. First, provide a fiber woven fabric which includes a polyacrylonitrile fiber. Next, carbonize the fiber woven fabric with high temperature to produce the carbonized material 100, wherein the fiber woven fabric is carbonized in an environment, which has a pressure between 100 torr and 1 atmospheric pressure and a temperature between 500 and 1200° C., for 1 to 25 minutes. In addition, an inert gas such as nitrogen is supplied to the environment during the carbonization process to block off oxygen in the air, so as to avoid oxidation reaction. The method of manufacturing the carbonized material 100 is not described in detail herein because it has been fully described in the abovementioned patent.

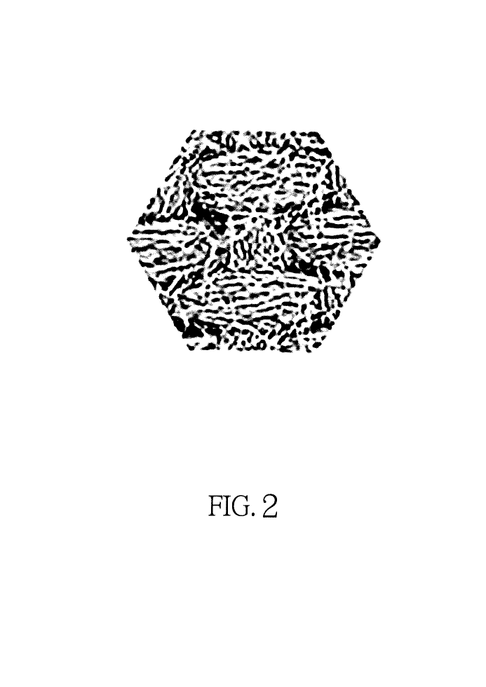

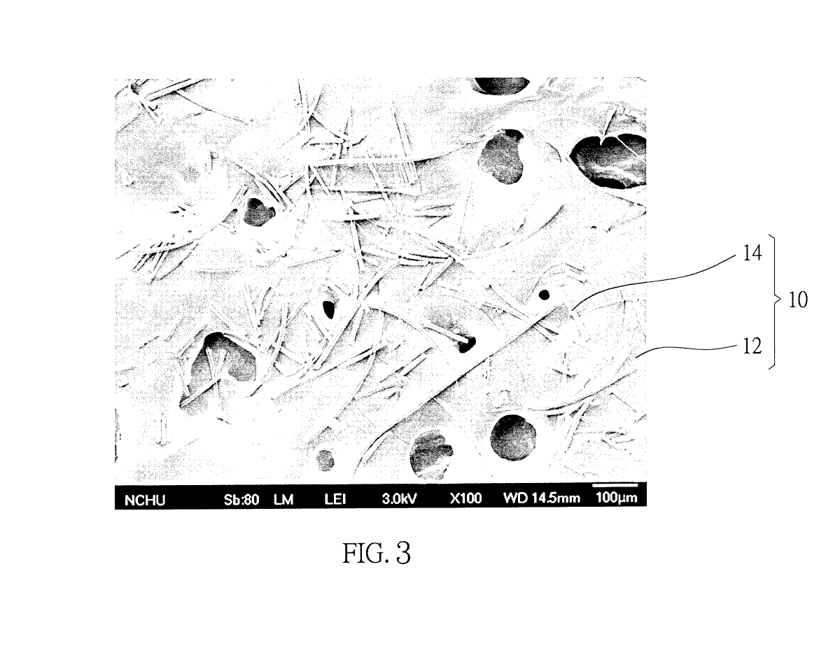

As shown in FIG. 2, the carbonized material 100 includes a plurality of carbon fascicles arranged in a crisscross pattern, wherein each of the plurality of carbon fascicles consists of a plurality of linear carbon fibers 10. The characteristic of the carbon fiber 10 is that, as shown in the scanning electron microscope (SEM) image at high magnification, each of the plurality of linear carbon fibers 10 includes a linear main body 12 and a plurality of pillars 14, wherein the plurality of pillars 14 are located on a surface of the main body 12, and are piled up with randomly arranged axes oriented in various directions (as shown in FIG. 3).

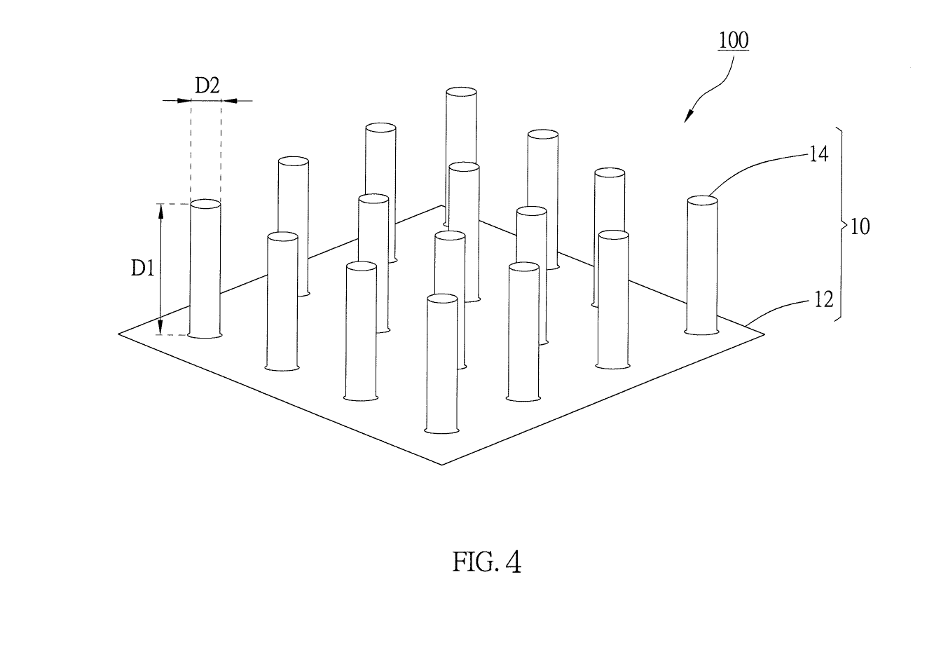

FIG. 4 may be referred to clearly define the size of the plurality of pillars and the relation between the pillars 14 and cells, wherein the pillars 14 are vertical to the surface of the main body 12, and are expressed in a matrix arrangement. The height D1 of each of the pillars 14 is substantially between 0.001 and 5 mm; the diameter D2 of each of the pillars 14 is substantially between 1 and 4 μm.

Finally, seed at least one cell 20 which has the ability to differentiate into at least one specialized cell on the carbonized material 100. In this preferred embodiment, the at least one cell 20 includes a plurality of cells 20, wherein the cells 20 are human neural stem cells (hNSCs), but this is not a limitation of the present invention. In other embodiments, the cells 20 may be neuron-like cells which are neural precursor cells also capable of differentiating into neurons. Each neuron has particular functions of growing an axon, and releasing dopamine through the axon to send electrical impulses as well as to deliver signals to another cell.

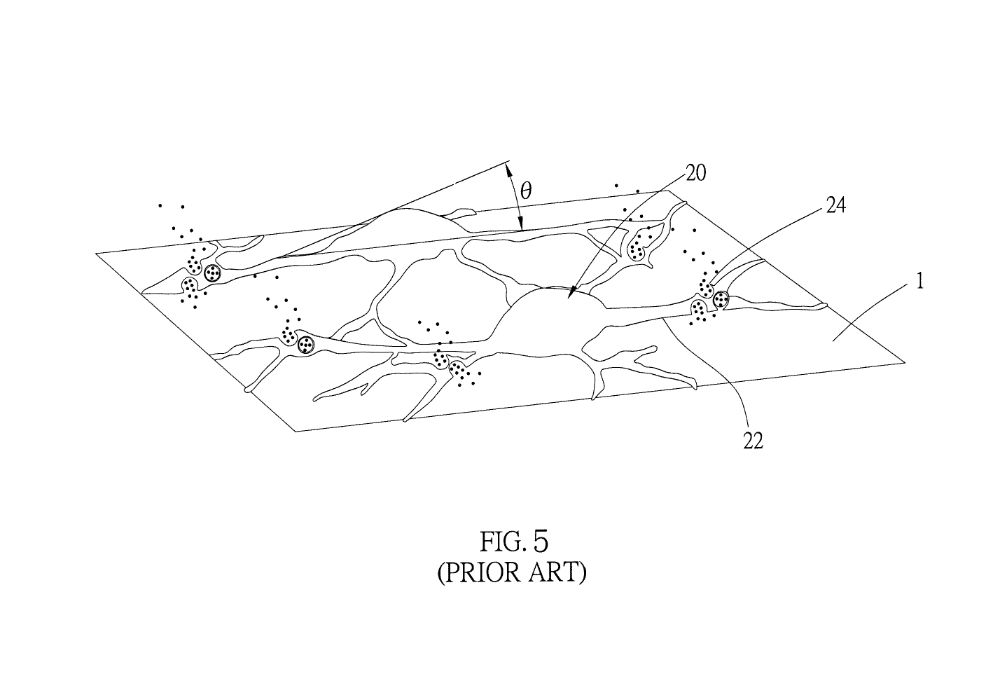

As shown in FIG. 5, hNSCs are typically cultured on a two-dimensional (2D) cell culture substrate 1 in the process of differentiation. As mentioned above, the differentiation rate of the cells 20 is low in this way. In other words, the speed of growing axons 22 of the cells 20 is slow, and the efficiency of releasing dopamine 24 is low, too. Additionally, because each of the cells 20 is flatly attached to the substrate 1, a contact angle θ between a topmost point of each of the cells 20 and a surface of the substrate 1 is small, as illustrated in FIG. 5. Small contact angle θ causes poor cell growth, slow movement, and low differentiation rate.

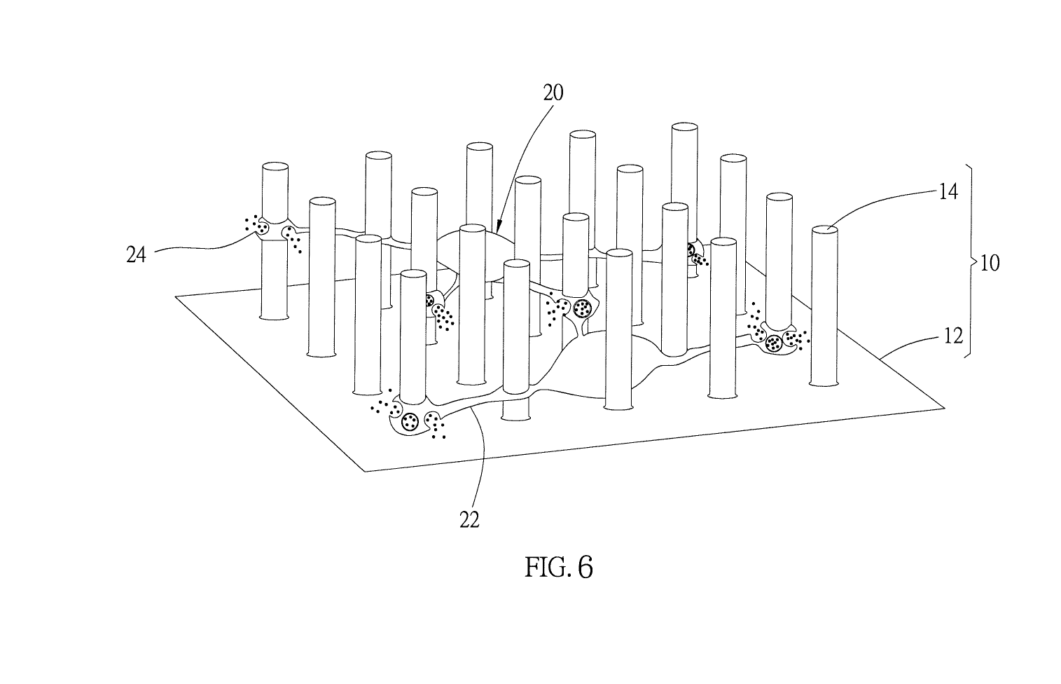

As shown in FIG. 6, in this preferred embodiment, the pillars 14 of the carbonized material 100 allow the cells 20 to attach thereon in a more 3D way, which is beneficial for the axons 22 generation and the release of dopamine 24 from the terminals of the axons 22. Moreover, the carbonized material 100 is electrically conductive, as the carbon atoms in the carbonized material 100 are in the form of sp² hybridization. Therefore, the carbon atoms in the carbonized material 100 are apt to accept electrons. On the other hand, dopamine 24 serves as an electron donor which is attracted by the pillars 14; thereby, the terminals of the axons 22 are able to firmly attach onto the pillars 14. With the relation between the axons 22 and the pillars 14, the cells 20 are not flatly attached to the surface of the main body 12, which may increase the contact angle θ, and may consequently advantage cell growth, movement, and differentiation.

Furthermore, cell differentiation not only causes morphological change and dopamine 24 release of the cells 20, but also increases the concentration of tyrosine hydroxylase in the cells 20. Tyrosine hydroxylase is an enzyme which catalyzes tyrosine hydroxylation, and is essential for dopamine 24 formation. Therefore, the concentration and the activation of tyrosine hydroxylase in the cells 20 may be seen as markers of cell differentiation efficiency. In comparison with the cells attaching on the substrate 1, the cells 20 seeded on the carbonized material 100 have higher concentration and activation of tyrosine hydroxylase after the process of cell differentiation. From the above, the carbonized material 100 indeed induces morphological change and cell signaling about cell differentiation so as to improve differentiation of the cells 20.

In conclusion, the carbonized material 100 serves as the substrate for cell differentiation, and induces cell growth and differentiation due to the three-dimensional structure and conductivity thereof.

The carbonized material 100 in this embodiment may further apply to a culture dish, wherein the culture dish includes a base and a containing space; the base has an upper surface which faces the containing space. The carbonized material 100 may be coated on the upper surface to be attached by the cells 20 to induce cell differentiation accordingly.

The carbonized material 100 may also be used for regenerating a nervous system which is deficient due to diseases or injuries. The tissue and blood near a region of injury have stem cells for tissue regeneration. Therefore, for the purpose of repairing the neurological deficiency, building a biological scaffold at the region of injury is essential for cell attachment, proliferation, and differentiation. However, conventional biological scaffolds may support tissue regeneration under a certain extent, but have no competence in inducing tissue regeneration and cell differentiation. Hence, if the carbonized material 100 serves as the biological scaffold at the region of injury, the growth and differentiation of the stem cells would be induced to achieve a better regeneration effect.

It must be pointed out that the embodiments described above are only some preferred embodiments of the present invention. All equivalent structures and methods which employ the concepts disclosed in this specification and the appended claims should fall within the scope of the present invention.

1. A method of inducing cell differentiation with a carbonized material, comprising:
providing a fiber woven fabric;
carbonizing the fiber woven fabric to produce a carbonized material, wherein the fiber woven fabric is carbonized in an environment having a pressure between 100 torr and 1 atmospheric pressure and a temperature between 500 and 1200° C. for 1 to 25 minutes, and wherein an inert gas is supplied to the environment during the carbonization process such that the carbonized material comprises a plurality of carbon fibers having a main body and a plurality of pillars, and wherein the plurality of pillars are located on a surface of the main body and are piled up with randomly arranged axes oriented in various directions within the carbonized material; and
seeding a cell having an ability to differentiate into a specialized cell on the carbonized material, wherein the specialized cell has a particular function, whereby through the attachment to the plurality of pillars during the carbonization process, the cell is induced to differentiate into the specialized cell;
wherein the specialized cell comprises a neuron;
wherein the particular function of the neuron is growing an axon, and releasing dopamine through the axon;
wherein the method is used for culturing a specialized cell for regenerating a deficient nervous system within the carbonized material.

2. The method of claim 1, wherein a diameter of each of the plurality of pillars is substantially between 1 and 4 m.

3. The method of claim 1, wherein a height of each of the plurality of pillars is substantially between 0.001 and 5 mm.

4. The method of claim 1, wherein the cell comprises a neural stem cell.

5. (canceled)

6. (canceled)

7. (canceled)

8. The method of claim 1, wherein the specialized cell enhances regeneration of neurons.

9. The method of claim 1, wherein the fiber woven fabric comprises a polyacrylonitrile fiber

10. A carbonized material as a substrate of cell differentiation, which is made by a method comprising:
providing a fiber woven fabric, which comprises a polyacrylonitrile fiber; and
carbonizing the fiber woven fabric with high temperature to produce a carbonized material, wherein the fiber woven fabric is carbonized in an environment, which has a pressure between 100 torr and 1 atmospheric pressure and a temperature between 500 and 1200° C., for 1 to 25 minutes; an inert gas is supplied to the environment during the carbonization process;
wherein the carbonized material comprises a plurality of carbon fibers, wherein each of the plurality of carbon fibers comprises a main body and a plurality of pillars; the plurality of pillars are located on a surface of the main body, and are piled up with axes thereof arranged in various directions.
